(19) United States
(12) Patent Application Publication
(10) Pub. No.: US 2022/0269227 A1
(43) Pub. Date: Aug. 25, 2022

(54) COMPUTER-READABLE RECORDING MEDIUM RECORDING CONTROL PROGRAM, INFORMATION PROCESSING APPARATUS AND CONTROL METHOD

(71) Applicant: FUJITSU LIMITED, Kawasaki-shi (JP)

(72) Inventor: Yoshinobu Iimura, Kawasaki (JP)

(73) Assignee: FUJITSU LIMITED, Kawasaki-shi (JP)

(21) Appl. No.: 17/548,544

(22) Filed: Dec. 12, 2021

(30) Foreign Application Priority Data
Feb. 25, 2021 (JP) 2021-028660

(51) Int. Cl. G05B 15/02

(52) U.S. Cl. CPC G05B 15/02

(57) ABSTRACT

A non-transitory computer-readable recording medium stores a control program causing a computer to execute a processing including: acquiring an actual result of a target that fluctuates according to a control by a system; calculating a weight for a control values to be input to the system according to a comparison between the actual result and a specific range; calculating the control value based on the actual result and the weight; and controlling the target by inputting the calculated control value to the system.

### CROSS-REFERENCE TO RELATED APPLICATION

This application is based upon and claims the benefit of priority of the prior Japanese Patent Application No. 2021-28660, filed on Feb. 25, 2021, the entire contents of which are incorporated herein by reference.

### FIELD

The embodiment discussed herein is related to a technical field of a control of a control target.

### BACKGROUND

There is a technique for controlling data to be controlled so as to be within a target range by using a prediction result by the model generated by machine learning or the like.

Japanese Laid-open Patent Publication No. 2006-172364, Japanese Laid-open Patent Publication No. 2013-142376 and US Patent Publication No. 2020/0015738 are disclosed as related art.

### SUMMARY

According to an aspect of the embodiments, a non-transitory computer-readable recording medium stores a control program causing a computer to execute a processing including: acquiring an actual result of a target that fluctuates according to a control by a system; calculating a weight for a control values to be input to the system according to a comparison between the actual result and a specific range; calculating the control value based on the actual result and the weight; and controlling the target by inputting the calculated control value to the system.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating a schematic structure of a control system;

FIG. 2 is a diagram for explaining an application example of a blood glucose level control by insulin administration;

FIG. 3 is a functional block diagram of a control device;

FIG. 4 is a diagram for explaining a case where an actual result of a target data does not fall within a target range;

FIG. 5 is a diagram for explaining a processing of a weight learning unit;

FIG. 6 is a diagram illustrating an example of a weight database (DB);

FIG. 7 is a diagram for explaining a processing of the weight learning unit;

FIG. 8 is a diagram for illustrating an example of a relationship between a plurality of yᵢₙᵢ and Λ accumulated in a weight DB;

FIG. 9 is a diagram for illustrating a schematic structure of a computer which functions as a control device;

FIG. 10 is a flowchart illustrating an example of a control process;

FIG. 11 is a flowchart illustrating an example of a weight learning process;

FIG. 12 is a diagram illustrating an example of a control result at the time of not learning and calculating a weight with respect to a control value;

FIG. 13 is a diagram illustrating a comparison between a predicted value and the actual result of the target data at the time of not learning and calculating the weight with respect to the control value; and

FIG. 14 is a diagram for illustrating an example of the control result by an embodiment.

### DESCRIPTION OF EMBODIMENTS

For example, a model estimation control device have been proposed which includes a feedback processing unit, feeds back a measured value of a state amount or a controlled amount of a control target, and corrects a search range or a predicted value in an optimum operation amount search unit and an estimated value in an internal state estimation unit by using the measured value. For example, this device sets a calculation range of an operation amount by an operation amount candidate calculation unit based on the measured value of the state amount or the controlled amount which is fed back to omit unnecessary calculation. In addition, this device corrects the operation amount candidate that is an input to a model prediction unit, the control amount that is an output, or an optimum control amount that is input to the control target, and further corrects the output from the internal state estimation unit to improve prediction accuracy.

Further, for example, a control device for an internal combustion engine has been proposed in which the calculation of the optimum operation amount is performed by model prediction control using a simple plant model. This device calculates the history of the predicted value of the controlled amount in the past predetermined period using a linear plant model from the history of a command value or an actual value of the operation amount in the past predetermined period. Further, this apparatus acquires the history of the actual value of the controlled amount in the past predetermined period, and calculates the parameter of the LPV error function based on the history of a difference between the predicted value and the actual value of the controlled amount in the past predetermined period. Then, this apparatus modifies the linear plant model by the LPV error function, and calculates the command value of the operation amount in the next step or a predetermined period after the next step by the model prediction control using the modified linear plant model.

For example, a system including a blood glucose level sensor, an insulin infusion device, and a control unit that predicts a future progress of the patient's blood glucose level from a physiological model and controls the insulin infusion device by considering the prediction. Has been proposed. The control unit performs steps of automatic calibration of the physiological model by considering the history of blood glucose levels measured by the sensor during a past observation period. At the end of the calibration step, the control unit judges whether the model is a sufficient one based on at least one numerical indicator that represents an error between the model-based estimated blood glucose level and the actual blood glucose level measured by the sensor. The control unit controls the insulin infusion device without considering the predictions made from the model if the quality of the model is not sufficient.

When controlling the data to be controlled so as to be within the target range by using the prediction result by the model, the data to be controlled may not be within the target range. One of the reasons for this is that the prediction of the model is not correct. In this case, in order to keep the data to be controlled within the target range, it is conceivable to refine the model to improve the prediction accuracy. However, in order to improve the prediction accuracy of the model, for example, in a case of refining the model by a statistical method, there is a problem in which a large amount of training data and a large amount of processing are required and a handling may not be performed during the control of the control target.

As one aspect, the disclosed technology aims to adaptively control the control target within a specific range.

Hereinafter, an example of the embodiment according to the disclosed technology will be described with reference to the drawings. In the following embodiment, the case of blood glucose control by insulin administration will be described as an application example of the disclosed technique, but the application example is not limited to this.

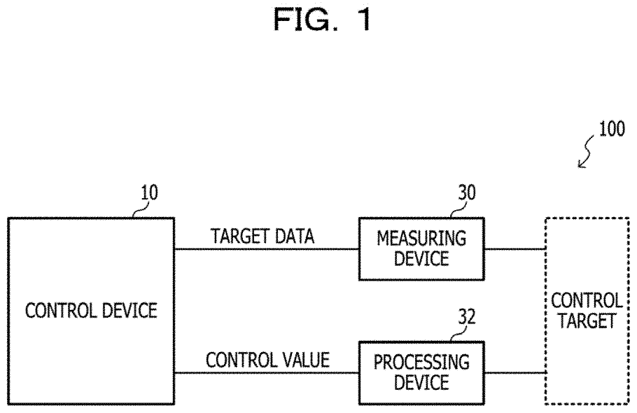

As shown in FIG. 1, the control system 100 according to the present embodiment includes a control device 10, a measuring device 30, and a processing device 32. The measuring device 30 measures and outputs data to be controlled (hereinafter, referred to as “target data”). The processing device 32 executes a predetermined process for controlling the control target based on a control value calculated by the control device 10. The control device 10 predicts future target data using the actual results of the target data, and calculates the control value to be input to the processing device 32 so that the target data falls within a target range based on the prediction result. The control target is an example of the “object” of the disclosed technology, and the target range is an example of the “specific range” of the disclosed technology.

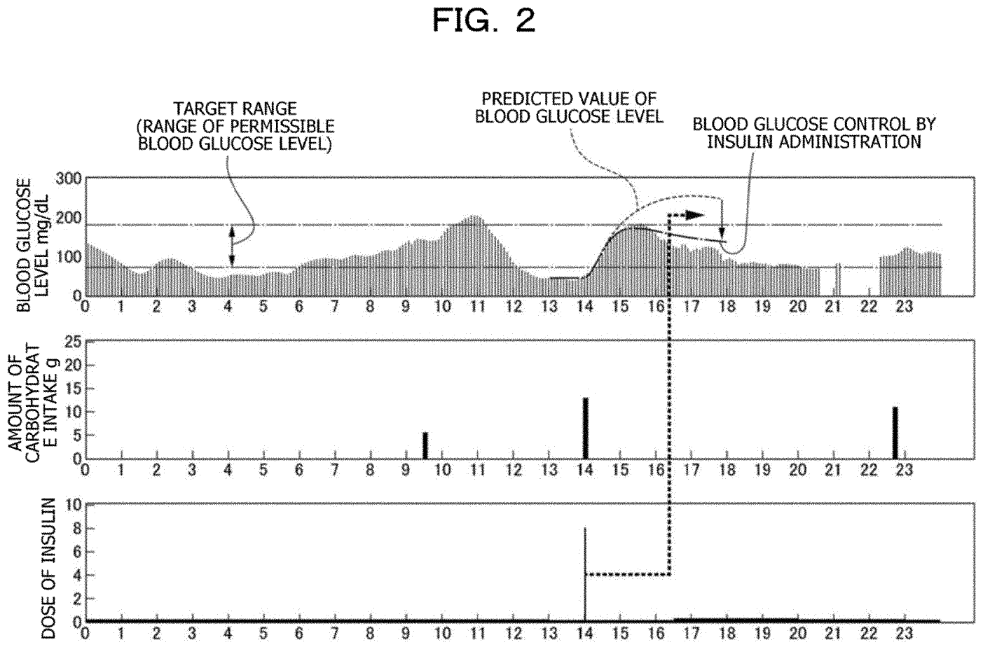

In the case of the application example of blood glucose control by insulin administration, the control target is the blood glucose level of the patient, the measuring device 30 is, for example, a blood glucose measuring device for measuring the blood glucose level of the patient, and the processing device 32 is, for example, an insulin pump that administers insulin to a patient. The blood glucose level of the patient fluctuates according to the insulin dose and the like. In this application example, for example, as shown in FIG. 2, when the predicted value of the blood glucose level predicted based on the actual blood glucose level exceeds an upper limit of the target range, the blood glucose level is lowered by administering insulin and control is performed so that the blood glucose level is within the target range. On the other hand, when the predicted value of the blood glucose level is below the lower limit of the target range, the blood glucose level is increased by reducing the dose of insulin, and the blood glucose level is controlled to be within the target range. For example, the control device 10 calculates a control value indicating the insulin dose based on the blood glucose level of the patient measured by the blood glucose level measuring device which is the measuring device 30. Then, based on the calculated control value, insulin is administered to the patient by the insulin pump which is the processing device 32. The bar graph in the upper part of FIG. 2 shows the actual results of the blood glucose level, which is the target data. Also, in this application, the target range is, for example, the range of permissible blood glucose levels. In addition, although FIG. 2 also includes data on the amount of carbohydrate intake that affects the blood glucose level, description thereof will be omitted here.

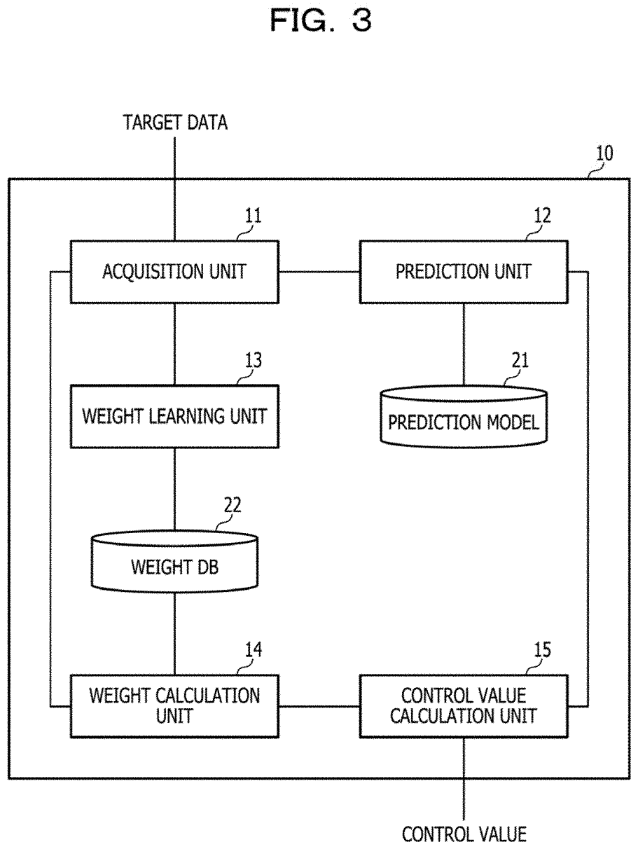

Functionally, as shown in FIG. 3, the control device 10 includes an acquisition unit 11, a prediction unit 12, a weight learning unit 13, a weight calculation unit 14, and a control value calculation unit 15. Further, the prediction model 21 and the weight DB (Database) 22 are stored in the predetermined storage area of the control device 10.

The acquisition unit 11 acquires the actual results of the target that fluctuates according to the control by the control system 100. For example, the acquisition unit 11 acquires the target data measured by the measuring device 30. The acquisition unit 11 passes the acquired target data to each of the prediction unit 12, the weight learning unit 13, and the weight calculation unit 14.

The prediction unit 12 predicts a value of the target data (hereinafter referred to as “predicted value”) at the time when the control target is controlled and after the time, based on the prediction model 21, the actual result of the target data passed from the acquisition unit 11, and the control value calculated by the control value calculation unit 15 described later. Hereinafter, the time when the control target is controlled is referred to as “control time”. Further, in the present embodiment, it is assumed that the calculation time of the control value, the time when the control value is input to the processing device 32, and the time when the process for controlling the control target by the processing device 32 is executed are the same. Those times are called the control time. The prediction model 21 is generated in advance by machine learning so as to output, when the actual result and the control value of the target data at a certain time are input, the predicted value of the target data at and after the certain time. For example, the prediction model 21 may output the predicted value under constraints of the following equations (1) and (2).

xₖ₊₁=Axₖ+Buₖ (1)

yₖ=Cxₖ (2)

k is an index indicating the time. In the following, the time indicated by the index k is referred to as “time k”. yₖ is the actual result of the target data at time k, uk is the control value at the time k, and xₖ is the predicted value of the target data at the time k. Further, A, B, and C are parameter matrices defined by machine learning. The prediction unit 12 uses the prediction model 21 to predict the predicted value of the target data at each of the plurality of times at and after the control time. For example, the prediction unit 12 sets a period of a long-term prediction as h and predicts the predicted value xₖ₊ᵢ (i=0, 1, . . . , h) of the target data at each of the times k, k+1, . . . , k+h . . . The prediction unit 12 passes the predicted value of the predicted target data to the control value calculation unit 15.

The control value calculation unit 15 calculates the control value based on the predicted value passed from the prediction unit 12 and the weight (details will be described later). For example, the control value calculation unit 15 calculates the control value is calculated by optimizing an objective function including a term for performing an optimization in which the predicted value of the target data falls within the target range and a term for optimizing an input plan of the control value. For example, the objective function may be the following equation (3).

$${{\sum }^{h}}_{i = 0} {x}_{k + i}^{T} Q {x}_{k + i} + {{\sum }^{N}}_{i = 0} {u}_{k + i}^{T} R {u}_{k + i} \qquad ( 3 )$$

The first term of the equation (3) is a term for performing the optimization in which the predicted value of the target data described above falls within the target range, and the second term is a term for optimizing the input plan of the control value. N represents a final input timing of the input plan of the control value between the times k and k+h. For example, when the control value is input at the times k, k+j and k+N, i in the second term is i=0, j and N. Q is the weight for the predicted value of the target data, R is the weight for the control value, and T is the transpose. The control value calculation unit 15 substitutes the predicted value xₖ₊ᵢ (i=0, 1, . . . , h) of the target data passed from the prediction unit 12 and the weight R passed from the weight calculation unit 14 (3) to the objective function of the equation (3), and calculates the control value uₖ₊ᵢ that minimizes the objective function.

The control value calculation unit 15 controls the control target by outputting the control value uₖ at the time k, which is the control time, from among the calculated control values uₖ₊ᵢ and inputting the control value uₖ to the processing device 32. Further, the control value calculation unit 15 passes the calculated control value uₖ₊ᵢ to the prediction unit 12 in order to use it for the long-term prediction of the target data at a next control time.

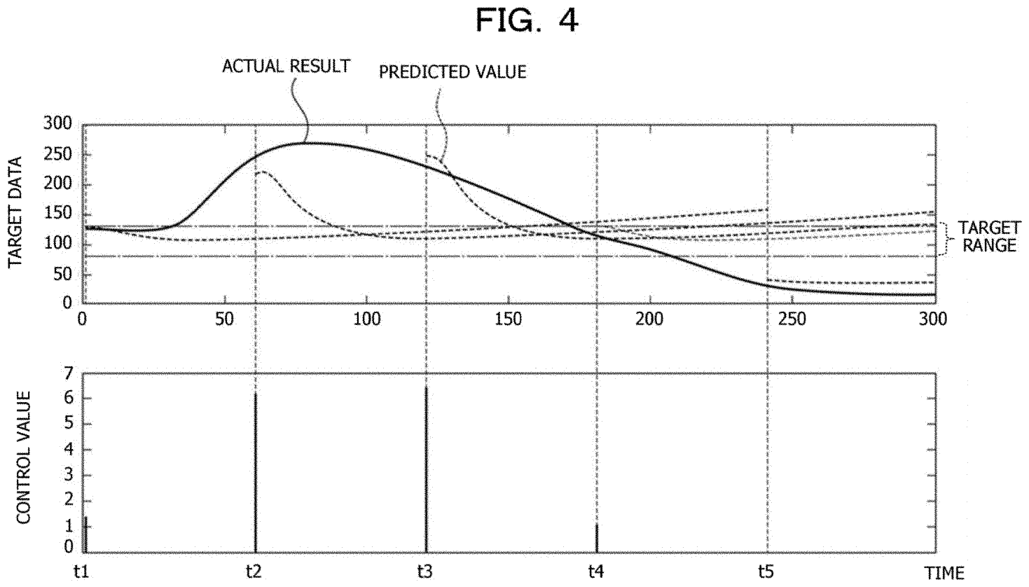

Here, if the control value calculated by the control value calculation unit 15 is not appropriate, as illustrated in FIG. 4, the actual result of the target data does not fall within the target range. In the example of FIG. 4, an example of the control target is illustrated in which the larger the control value, the lower the value of the target data. For example, it is such an example in which the blood glucose level, which is the control target, is lowered by the administration of insulin, such as the blood glucose level control by insulin administration. In this example, it is considered that the control value input at the time t3 is too large, so that the actual result of the target data becomes too low and greatly lowers from the target range. In this way, it is considered that the reason why the control value in which the actual result of the target data exceeds the target range is calculated is that the long-term prediction of the target data is not correct.

Therefore, as a method simply assumed in order to make the target data within the target range, it is conceivable to improve the accuracy of the long-term prediction of the target data by the prediction unit 12. In order to improve the prediction accuracy, for example, it is conceivable to refine the prediction model 21 by a statistical approach. However, this approach requires a large amount of training data to perform machine learning for the predictive model 21. For example, in the case of the prediction model 21 constrained by the equations (1) and (2), the number of parameters to be determined is the total number of each element of the parameter matrices A, B, and C. For example, when xk is four-dimensional, A is a 4×4 element, B is a 4×1 element, and C is a 4×1 element so that it is necessary to determine a total of 24 parameters. It takes some time to collect such a large amount of training data for determining many parameters. Depending on the control target, it may be desirable to respond adaptively before a sufficient number of training data are collected. For example, in the application example of blood glucose control by insulin administration, when the blood glucose level exceeds the target range on the hyperglycemic side, arteriosclerosis may cause cerebral infarction, myocardial infarction, necrosis and the like. In addition, if the blood glucose level exceeds the target range on the hypoglycemic side, hypoglycemia may directly lead to a life-threatening situation. In order to avoid such a situation, it is necessary to take adaptive measures to make the target data within the target range.

Therefore, the control device 10 according to the present embodiment adaptively corrects the control value directly through an optimization problem of calculating the control value based on the past control results before the current control time. For example, the control device 10 corrects the weight for the control value in the optimization, for example, R in the case of the objective function of the equation (3). As described above, since the control value uₖ which is too large or too small causes the target data to be out of the target range, the uₖ is adjusted via R. For example, in the example of the equation (3), the control device 10 corrects R by utilizing the fact that uₖ decreases as R increases and uₖ increases as R decreases. In this case, since there is only one parameter to be corrected, an adaptive response is possible as compared with the case where the prediction model 21 is refined by the statistical approach as described above. Hereinafter, each of the weight learning unit 13 and the weight calculation unit 14 regarding the weight calculation will be described in detail.

The weight learning unit 13 learns the relationship between the actual result of the target data at a predetermined time and the weight corresponding to the actual result. For example, this relationship is for determining the weight for the control value to be calculated at the predetermined time based on the actual result of the target data at that time. The weight learning unit 13 uses a weight which is corrected based on the actual result for a predetermined period from the predetermined time as the weight defined in this relationship. For example, the weight learning unit 13 specifies, for each control time, the weight associated with a value of the actual result which is closest to an actual result at a time before the predetermined period of the control time and the actual result of the value closest to the actual result at a time before the predetermined period of the control time in the already learned relationship. Then, the weight learning unit 13 corrects the specified weight according to a comparison between the actual result for the predetermined period from the predetermined time and the target range, and adds the correspondence between the corrected weight and the time before the predetermined period of the control time to the already learned relationship.

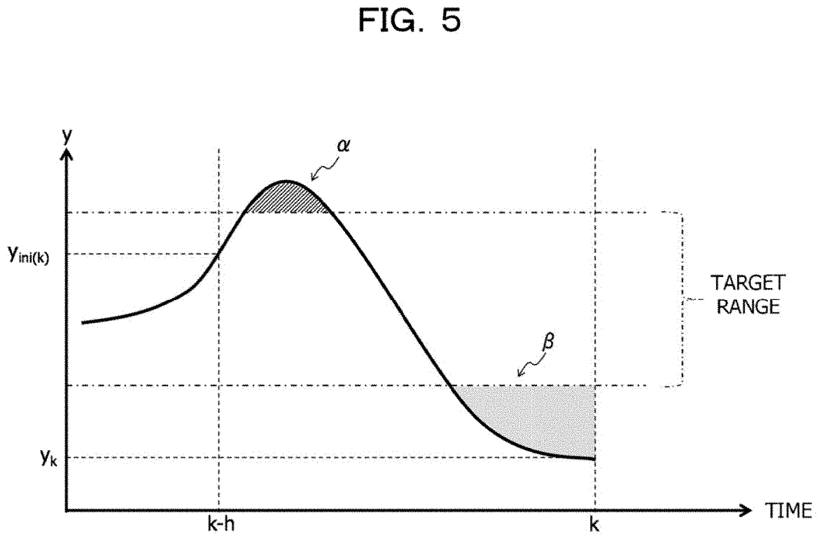

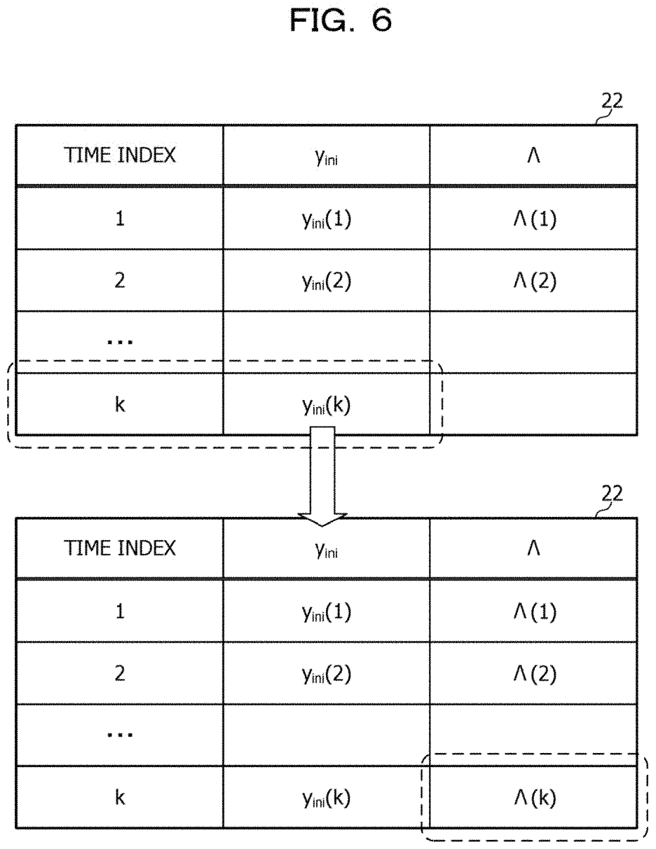

The processing of the weight learning unit 13 will be described more specifically. As illustrated in FIG. 5, the weight learning unit 13 sets the actual result of the target data at the time k-h which is one cycle (h) before the control time k as yᵢₙᵢ (k) and the actual result of the target data at the time k as yₖ. Then, the weight learning unit 13 stores yᵢₙᵢ (k) in the weight DB 22 in association with the time index k. FIG. 6 illustrates an example of the weight DB 22. In the example of FIG. 6, the weight DB 22 stores yᵢₙᵢ and the weight Λ at that time in association with the time index k. The weight in the weight DB 22 uses the symbol “Λ (lambda)” to distinguish it from the weight R used for calculating the control value at the control time. Here, as shown by the broken line portion in the upper part of FIG. 6, k and yᵢₙᵢ (k) are stored in the weight DB 22 in association with each other.

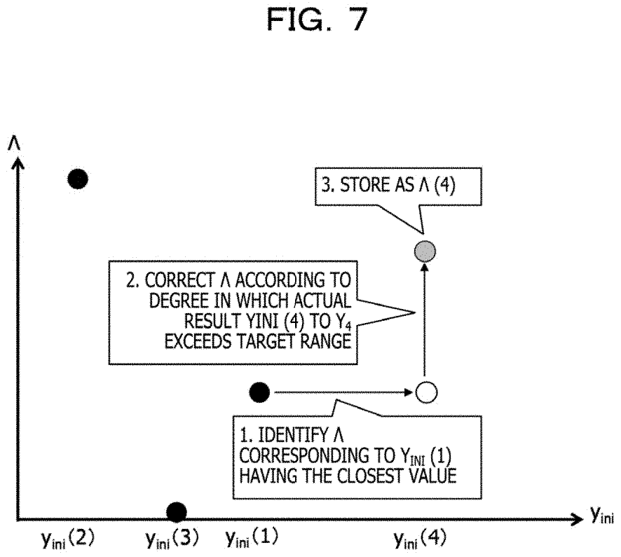

The weight learning unit 13 identifies the weight Λ (ind1) corresponding to the yᵢₙᵢ (ind1) having the closest value to the yᵢₙᵢ (k) among the yᵢₙᵢ stored in the weight DB 22. Note that ind1 is the time index corresponding to yᵢₙᵢ, which is the closest value to yᵢₙᵢ (k). For example, FIG. 7 illustrates a relationship between the yᵢₙᵢ (k) and Λ (k) when the control time k is 4 (k=4) and the weights DB 22 already stores the yᵢₙᵢ (k) and Λ (k) of k=1, 2, and 3. In FIG. 7, black circles represent the relationship between yᵢₙᵢ (k) and Λ (k) (k=1, 2, 3) which are already stored in the weight DB 22. In this case, the weight learning unit 13 identifies the Λ (1) corresponding to the yᵢₙᵢ (1) having the closest value to the yᵢₙᵢ (4) as shown by the white circle in FIG. 7, and once adopts it as the Λ (4).

As illustrated in FIG. 5, the weight learning unit 13 calculates an index α indicating a degree in which the actual result of the target data during the period from time k-h to time k exceeds the upper limit of the target range and an index β indicating the degree in which the actual result of the target data during the period from time k-h to time k exceeds the lower limit of the target range. The index α may be a value corresponding to the area of the shaded portion in FIG. 5. The index β may be a value corresponding to the area of the shaded portion in FIG. 5. For example, as shown in the following equation (4), the index α is the root mean square error of the actual result which exceeds the upper limit U of the target range from among the actual result yᵣ of the target data at each time r in the period from time k-h to time k. Similarly, as shown in the following equation (5), the index β may be the root mean square error of the actual results which is below the lower limit R of the target range from among the actual result yᵣ of the target data.

α=∥yᵣ (yᵣ>U)−U∥ (4)

β=∥L−yᵣ (yᵣ<L)∥ (5)

Note that, yᵣ (yr>U) of the formula (4) represents yᵣ above the upper limit U, and yᵣ (yᵣ<L) of the formula (5) represents yᵣ below the lower limit L.

The weight learning unit 13 corrects the specified Λ (ind1) based on the calculated index α and the calculated index β, and calculates Λ (k) for storing in the weight DB 22. For example, the weight learning unit 13 corrects Λ (ind1) so that the target data becomes smaller according to the size of the index α and the target data becomes larger according to the size of the index. Λ (ind1) is corrected to. When the target data becomes smaller as the control value becomes larger like in the application example of blood glucose control by insulin administration, the weight learning unit 13 corrects Λ (ind1) so as to become smaller in order to increase the control value as the index α becomes larger and calculates Λ (k). Similarly, the weight learning unit 13 corrects Λ (ind1) so as to become larger in order to decrease the control value as the index β becomes larger and calculates Λ (k).

For example, when α is larger than β (including the case where β is 0), the weight learning unit 13 may calculate the corrected Λ (k) by the following equation (6). Further, when β is larger than α (including the case where α is 0), the weight learning unit 13 may calculate the corrected Λ (k) by the following equation (7). Further, the weight learning unit 13 uses Λ (ind1) as it is as Λ (k), as described in equation (8) below, when both α and β are 0, for example, when the actual yᵣ of the target data at each time r in the period from time k-h to time k is within the target range.

When α>β≥0 Λ(k)=Λ(ind)+(α−β)/N1x(0−Λ(ind1)) (6)

When β>α≥0 Λ(k)=Λ(ind)+(β−α)/N2x(Rmax−Λ(ind1)) (7)

When α=0&β=0 Λ(k)=Λ(ind) (8)

Each of N1 and N2 is a standardized constant. Further, Rmax is a maximum value that is able to be set as the weight R. The above calculation method and case classification of Λ (k) are examples, and these may be appropriately changed according to the nature of the control target and the like. For example, in the case of application example of blood glucose control by insulin administration, there is a high risk in a case of hypoglycemia, for example, in a case where the target data falls below the target range. Therefore, even if α>β, the following equation (9) may be adopted if β>0.

Λ(k)=Λ(ind)+β/N2x(Rmax−Λ(ind1)) (9)

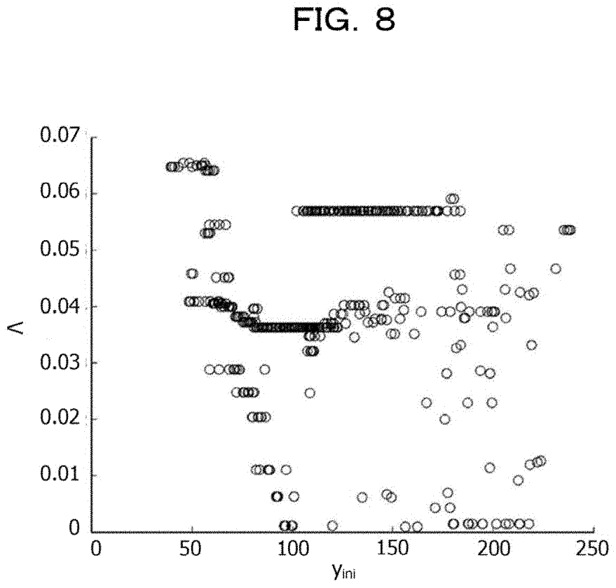

As illustrated in FIG. 7, in the case of the above-mentioned example of the control time k=4, the weight learning unit 13 corrects the specified Λ (1) (white circle in FIG. 7) according to the degree in which the actual result yᵢₙᵢ (4) to yₖ of the target data exceeds the target range, and calculates Λ (4) (shaded circle in FIG. 7). The weight learning unit 13 stores the calculated Λ (k) in the weight DB 22 in association with the time index k, as illustrated by the broken line portion in the lower part of FIG. 6. By repeating the above processing every the control time k by the weight learning unit 13, a plurality of relationships between yᵢₙᵢ and Λ are accumulated in the weight DB 22. FIG. 8 illustrates an example of the relationship between yᵢₙᵢ and Λ accumulated in the weight DB 22. In the example of FIG. 8, one circle represents one relationship between one yᵢₙᵢ and Λ.

Here, it is conceivable to express the relationship between yᵢₙᵢ and Λ by Λ=f (yini) using the function f (⋅). However, yᵢₙᵢ may only be obtained with a finite number of trials, and all continuous values may not be obtained. Moreover, it is unclear what kind of function should be prepared as f (⋅) because there is no prerequisite knowledge. If the appropriate function f (⋅) is not used, the corrected Λ may not be calculated appropriately, resulting in a decrease in control performance. On the other hand, as described above, the control device 10 according to the present embodiment corrects Λ for the past yᵢₙᵢ, which has the closest value to yᵢₙᵢ (k) at the control time k, based on the actual result of the target data, and stores the relationship between yᵢₙᵢ and corrected Λ. Then, the control device 10 may express an arbitrary f (⋅) by repeating the process described above every the control time k, and mayn suppress the deterioration of the control performance.

The weight calculation unit 14 calculates the weight corresponding to the actual result acquired this time based on the relationship between the actual result of the past target data and the weight corresponding to the actual result of the past target data which are stored in the weight DB 22. For example, the weight calculation unit 14 calculates the weight Λ (ind2) corresponding to the yini (ind2) having the value closest to the actual result yₖ of the target data at the control time k among the yᵢₙᵢ stored in the weight DB 22 as the weight R used to calculate the value uₖ. Ind2 is the time index corresponding to yᵢₙᵢ, which is the closest value to yₖ. This corresponds to calculating the weight R according to the target data at times k to k+h based on the past control result.

The weight calculation unit 14 passes the calculated weight R to the control value calculation unit 15. As a result, as described above, the control value calculation unit 15 calculates the control value using the weight R. The weight R is selected from the weight Λ stored in the weight DB 22, and this Λ is corrected by comparing the actual result of the target data with the target range. Therefore, by calculating the control value using the weight R, it is possible to control the target data so as to be within the target range.

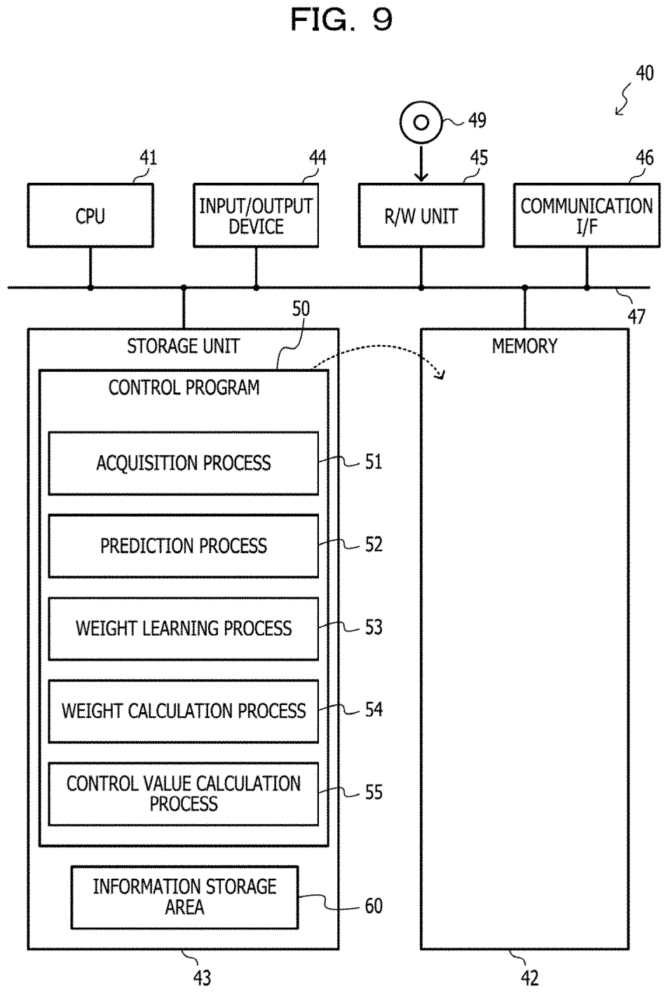

The control device 10 may be realized by, for example, the computer 40 illustrated in FIG. 9. The computer 40 includes a Central Processing Unit (CPU) 41, a memory 42 as a temporary storage area, and a non-volatile storage unit 43. Further, the computer 40 includes an input/output device 44 such as an input unit and a display unit, and an R/W (Read/Write) unit 45 that controls reading and writing of data from and to the storage medium 49. Further, the computer 40 includes a communication I/F (Interface) 46 connected to a network such as the Internet. The CPU 41, the memory 42, the storage unit 43, the input/output device 44, the R/W unit 45, and the communication I/F 46 are connected to each other via the bus 47.

The storage unit 43 may be realized by a Hard Disk Drive (HDD), a Solid State Drive (SSD), a flash memory, or the like. A control program 50 for causing the computer 40 to function as the control device 10 is stored in the storage unit 43 as a storage medium. The control program 50 includes an acquisition process 51, a prediction process 52, a weight learning process 53, a weight calculation process 54, and a control value calculation process 55. Further, the storage unit 43 has an information storage area 60 in which information constituting each of the prediction model 21 and the weight DB 22 is stored.

The CPU 41 reads the control program 50 from the storage unit 43, expands it in the memory 42, and sequentially executes the processes included in the control program 50. By executing the acquisition process 51, the CPU 41 operates as the acquisition unit 11 illustrated in FIG. 3. Further, the CPU 41 operates as the prediction unit 12 illustrated in FIG. 3 by executing the prediction process 52. Further, the CPU 41 operates as the weight learning unit 13 illustrated in FIG. 3 by executing the weight learning process 53. Further, the CPU 41 operates as the weight calculation unit 14 illustrated in FIG. 3 by executing the weight calculation process 54. Further, the CPU 41 operates as the control value calculation unit 15 illustrated in FIG. 3 by executing the control value calculation process 55. Further, the CPU 41 reads information from the information storage area 60 and expands each of the prediction model 21 and the weight DB 22 into the memory 42. As a result, the computer 40 that executes the control program 50 functions as the control device 10. The CPU 41 that executes the program is hardware.

The function realized by the control program 50 may also be realized by, for example, a semiconductor integrated circuit, for example, an Application Specific Integrated Circuit (ASIC) or the like.

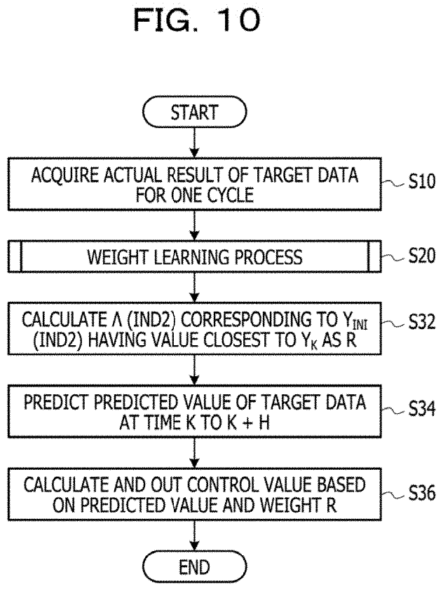

Next, the operation of the control system 100 according to the present embodiment will be described. When the measuring device 30 starts the measurement and output of the target data, the control device 10 executes the control process illustrated in FIG. 10 every the control time k. The control process is an example of a control method of the disclosed technology. In the following control process, a case where the target data is controlled to be smaller as the control value is larger will be described as an example, as in the case of blood glucose level control by insulin administration.

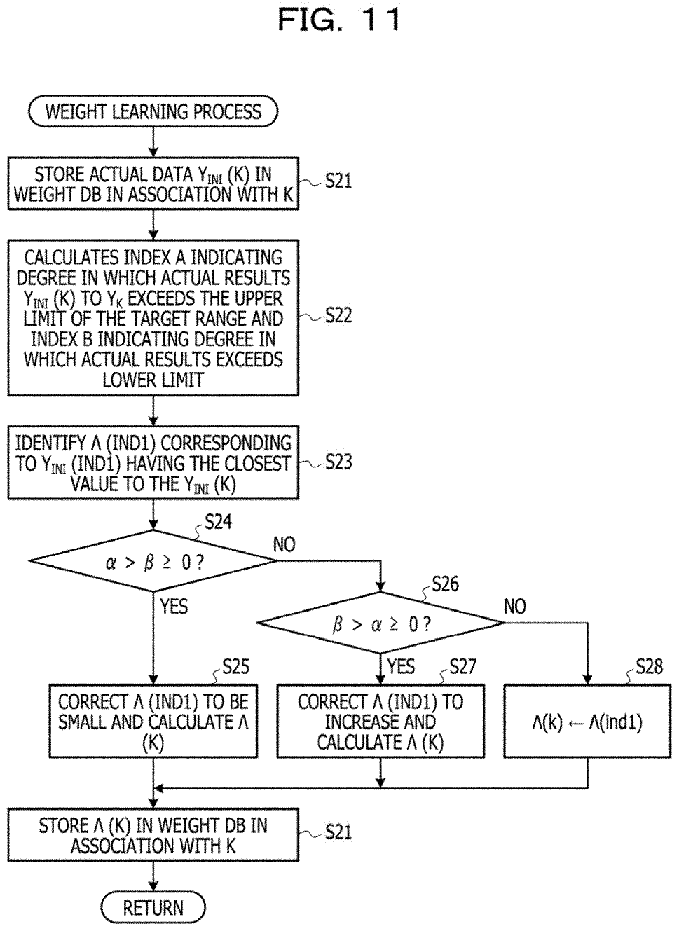

In step S10, the acquisition unit 11 acquires the actual result of the target data for one cycle of the long-term prediction of the target data, for example, at each time from time k-h to time k. Next, in step S20, the weight learning unit 13 executes the weight learning process. Here, the weight learning process will be described with reference to FIG. 11.

In step S21, the weight learning unit 13 stores, in the weight DB 22, the actual data yᵢₙᵢ (k) of the target data at the time k-h in association with the time index k among the actual result of the target data acquired in step S10 described above.

Next, in step S22, the weight learning unit 13 calculates an index α indicating the degree in which the actual results yᵢₙᵢ (k) to yₖ of the target data in the period from time k-h to time k exceeds the upper limit of the target range and the index β indicating the degree in which the actual results yᵢₙᵢ (k) to yₖ exceeds the lower limit of the target range. Next, in step S23, the weight learning unit 13 identifies the weight Λ (ind1) corresponding to the yᵢₙᵢ (ind1) having the closest value to the yᵢₙᵢ (k) among the yᵢₙᵢ stored in the weight DB 22.

Next, in step S24, the weight learning unit 13 determines whether or not β is 0 or more and α is larger than β. For example, it is determined whether or not the degree in which the actual data exceeds the upper limit of the target range is large. When α>β≥0, the process proceeds to step S25, and when α≤β, the process proceeds to step S26. In step S25, the weight learning unit 13 corrects Λ (ind1) to be small in order to increase the control value, and calculates Λ (k).

On the other hand, in step S26, the weight learning unit 13 determines whether or not αis 0 or more and β is larger than α. For example, it is determined whether or not the degree in which the actual data exceeds the lower limit of the target range is large. When β>α≥0, the process proceeds to step S27, and when α=β=0, the process proceeds to step S28. In step S27, the weight learning unit 13 corrects Λ (ind1) to increase in order to decrease the control value, and calculates Λ (k).

In step S28, the weight learning unit 13 sets Λ (ind1) as it is to Λ (k). When α=β>0, if the risk in which the target data exceeds the upper limit of the target range is large, it may be determined in advance that the weight learning unit 13 executes the process of step S25. Just do it. Further, when the risk in which the target data exceeds the lower limit of the target range is large, it may be determined in advance that the weight learning unit 13 executes the process of step S27. Further, when the risk in which the target data exceeds the upper limit and the risk in which the target data exceeds the lower limit are equal, the weight learning unit 13 may execute the process of step S28.

Next, in step S29, the weight learning unit 13 stores the Λ (k) calculated in step S25, S27, or S28 in the weight DB 22 in association with the time index k and ends the weight learning process. The process returns to the control process (FIG. 10).

Next, in step S32, the weight calculation unit 14 calculates the weight Λ (ind2) corresponding to the yᵢₙᵢ (ind2) having the value closest to the actual result yₖ of the target data at the control time k among the yᵢₙᵢ stored in the weight DB 22 as the weight R used to calculate the control value uₖ. Next, in step S34, the prediction unit 12 uses the actual result of the target data acquired in step S10 and the control value calculated at the previous control time and predicts the predicted value xₖ₊ᵢ (i=0, 1, . . . , h) of the target data at time k, k+1, k+h, respectively.

Next, in step S36, the control value calculation unit 15 calculates the control value uₖ₊ᵢ based on the predicted value xₖ₊ᵢ predicted in step S34 and the weight R calculated in step S32. Then, the control value calculation unit 15 controls the control target by inputting the uₖ of the calculated control values uₖ₊ᵢ to the processing device 32. Further, the control value calculation unit 15 passes the calculated control value uₖ₊ᵢ to the prediction unit 12 in order to use it for the long-term prediction of the target data at the next control time, and the control process ends.

As described above, according to the control system according to the present embodiment, the control device acquires the actual result of the target that fluctuates according to the control by the system, and calculates the weight for the control value which inputs according to the comparison between the actual result and the specific range. Then, the control system calculates the control value based on the actual result and the weight, inputs the calculated control value to the system, and controls the target. As a result, even if the prediction of the target data using the prediction model is incorrect, it is possible to adaptively perform the control in which the control target falls within the specific range without refining the prediction model.

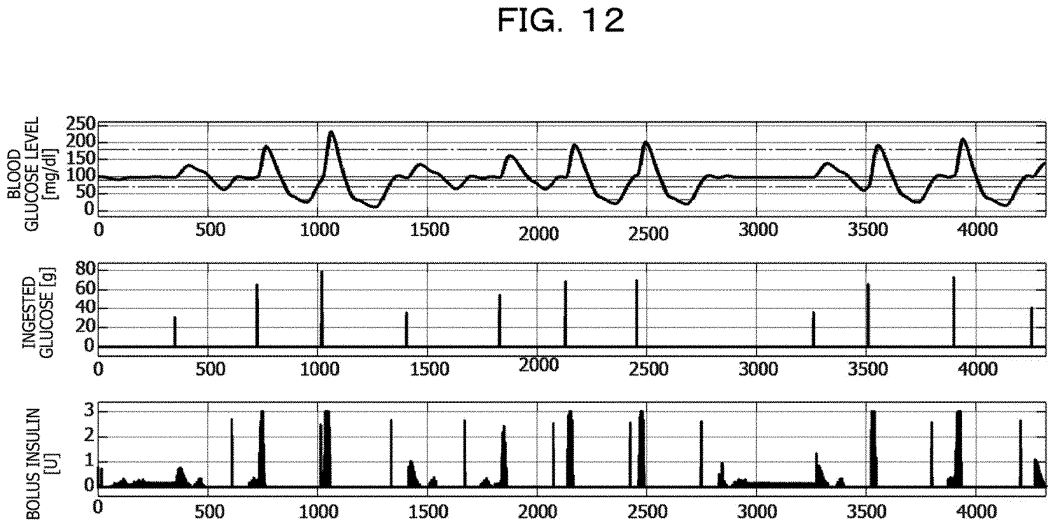

Here, the control result when the disclosed technique is applied to the blood glucose level control by insulin administration will be described. First, as a comparison, FIG. 12 illustrates an example of the control result when the weights for the control values are not learned and calculated. In FIG. 12, the blood glucose level is an example of the target data, and the bolus insulin is an example of the control value. In addition, in FIG. 12, the ingested glucose is the amount of glucose ingested by a meal, and is a factor that affects the fluctuation of the blood glucose level. The same applies to the amount of carbohydrate intake in FIG. 2 described above. In this way, when there is a factor that affects the fluctuation of the target data other than the control value, the equation (1) which is the constraint in the above prediction model may be changed to the equation in which the value zk of another factor is taken into account as in the following equation (10). Note that D is a parameter matrix.

xₖ₊₁=Axₖ+Buₖ+Dzₖ (10)

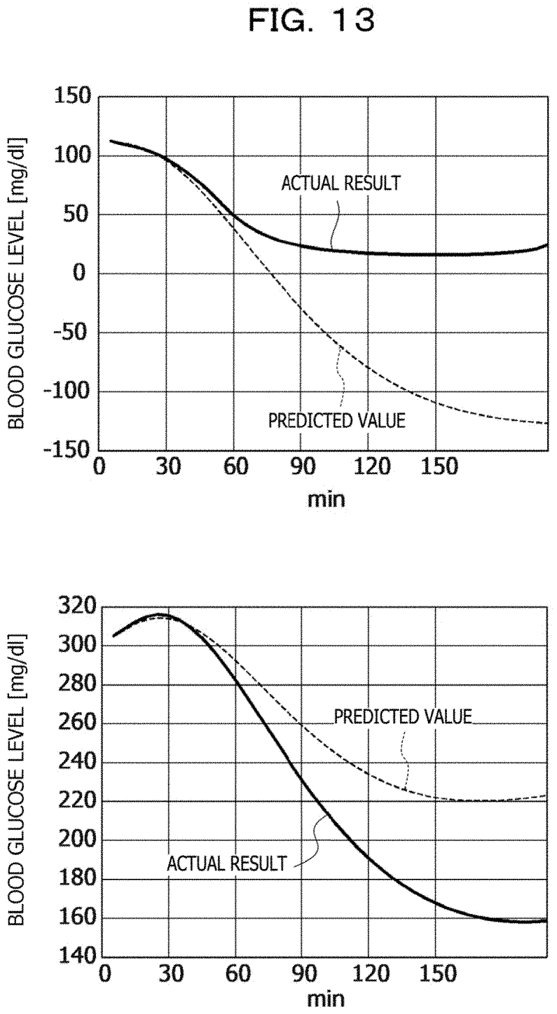

In FIG. 12, there are many time periods when the blood glucose level, which is the target data, is out of the target range, for example, when the blood glucose level is below the lower limit of the target range. In the example of FIG. 12, FIG. 13 illustrates a comparison between the predicted value of the target data predicted at each of the two times and the actual result. At both of the two times, there is a large discrepancy between the predicted value and the actual result, and it is considered that this is the cause of the blood glucose level, which is the target data, being out of the target range.

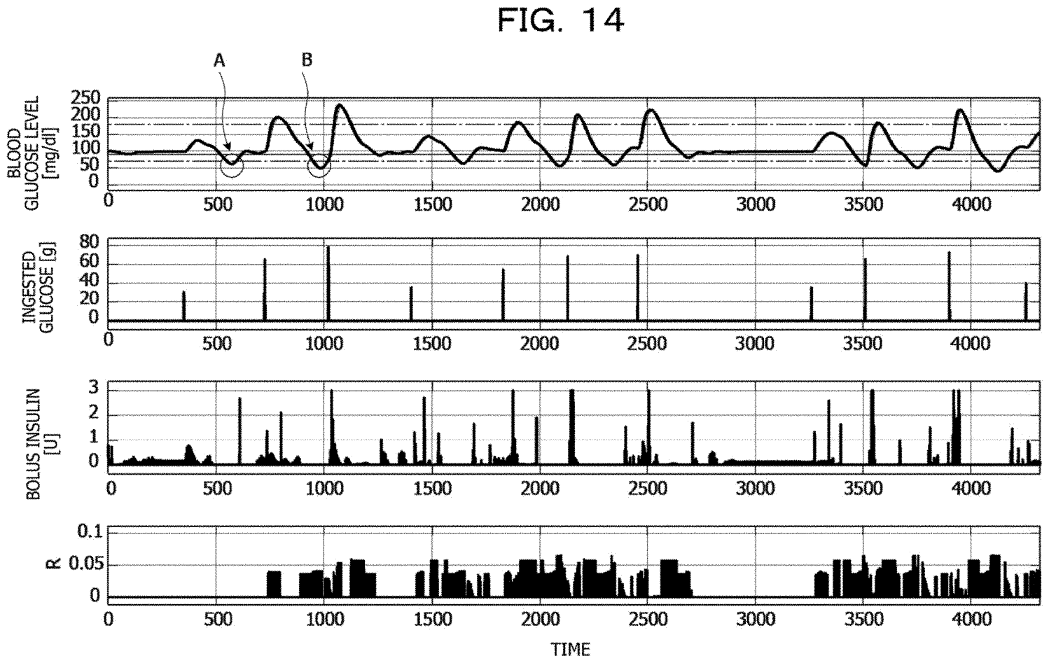

FIG. 14 illustrates an example of the control result when the weights for the control values are learned and calculated as in the present embodiment. It may be seen that the time period and the degree in which the blood glucose level, which is the target data, is out of the target range are reduced as compared with the example of FIG. 12. For example, in the example of FIG. 14, in the portion illustrated by A, correction is performed so as to increase the weight Λ corresponding to this portion based on the fact that the actual result of the target data falls below the target range. Then, by using this Λ in the subsequent calculation of the weight R, the insulin dose may be suppressed, and the degree of hypoglycemia may be reduced, for example, as illustrated by B.

In the above embodiment, when the application example is described, an example of blood glucose level control by insulin administration has been described, but the disclosed technique may be applied to other control systems such as an engine control.

Further, in the above embodiment, the mode in which the control program is stored (installed) in the storage unit in advance has been described, but the present embodiment is not limited to this. The program according to the disclosed technique may also be provided in a form stored in a storage medium such as a CD-ROM, a DVD-ROM, or a USB memory.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a control program causing a computer to execute a processing, the processing comprising:
acquiring an actual result of a target that fluctuates according to a control by a system;
calculating a weight for a control values to be input to the system according to a comparison between the actual result and a specific range;
calculating the control value based on the actual result and the weight; and
controlling the target by inputting the calculated control value to the system.

2. The non-transitory computer-readable recording medium according to claim 1, wherein
a process of the calculating the weight includes calculating the weight corresponding to the actual result which is acquired this time based on a relationship between a past actual result and a past weight corresponding to the past actual result.

3. The non-transitory computer-readable recording medium according to claim 2, wherein
the relationship is a relationship in which the actual result at a predetermined time and a weight which is calculated based on the actual result for a predetermined period from the predetermined time are associated with each other.

4. The non-transitory computer-readable recording medium according to claim 3, further comprising:
adding, for each control time when the target is controlled, correspondence between the actual result at a time before the predetermined period of the control time and a corrected weight to the relationship,
wherein the corrected weight is generated by correcting the weight associated with the actual result having the closest value to the actual result at the time before the predetermined period of the control time in the relationship based on a comparison between the actual result for the predetermined period from the time before the predetermined period of the control time and the specific range.

5. The non-transitory computer-readable recording medium according to claim 4, wherein
the process of the calculating the weight includes acquiring, in the relationship, the weight associated with the actual result closest to the actual result which is acquired this time.

6. The non-transitory computer-readable recording medium according to claim 1, wherein
a process of the calculating the control value includes calculating the control value based on a predicted value of the target according to the actual result and the weight.

7. The non-transitory computer-readable recording medium according to claim 6, wherein
the process of calculating the control value includes predicting the predicted value based on the model which is generated by machine learning, the actual result and the control value.

8. The non-transitory computer-readable recording medium according to claim 6, wherein
the process of calculating the control value includes optimizing an objective function including a term to optimize the predicted value of the target so as to fall within the specific range and a term to optimize an input plan of the control value.

9. An information processing apparatus comprising:
a memory; and
a processor coupled to the memory and configures to:
acquire an actual result of a target that fluctuates according to a control by a system;
calculate a weight for a control values to be input to the system according to a comparison between the actual result and a specific range;
calculate the control value based on the actual result and the weight; and
control the target by inputting the calculated control value to the system.

10. The information processing apparatus according to claim 9, wherein
a process to calculate the weight includes calculating the weight corresponding to the actual result which is acquired this time based on a relationship between a past actual result and a past weight corresponding to the past actual result.

11. The information processing apparatus according to claim 10, wherein
the relationship is a relationship in which the actual result at a predetermined time and a weight which is calculated based on the actual result for a predetermined period from the predetermined time are associated with each other.

12. The information processing apparatus according to claim 11, wherein:
the processor adds, for each control time when the target is controlled, correspondence between the actual result at a time before the predetermined period of the control time and a corrected weight to the relationship,
wherein the corrected weight is generated by correcting the weight associated with the actual result having the closest value to the actual result at the time before the predetermined period of the control time in the relationship based on a comparison between the actual result for the predetermined period from the time before the predetermined period of the control time and the specific range.

13. The information processing apparatus according to claim 12, wherein
the process to calculate the weight includes acquiring, in the relationship, the weight associated with the actual result closest to the actual result which is acquired this time.

14. The information processing apparatus according to claim 9, wherein
a process to calculate the control value includes calculating the control value based on a predicted value of the target according to the actual result and the weight.

15. The information processing apparatus according to claim 14, wherein
the process to calculate the control value includes predicting the predicted value based on the model which is generated by machine learning, the actual result and the control value.

16. The information processing apparatus according to claim 14, wherein
the process to calculate the control value includes optimizing an objective function including a term to optimize the predicted value of the target so as to fall within the specific range and a term to optimize an input plan of the control value.

17. A control method comprising:
acquiring, by a computer, an actual result of a target that fluctuates according to a control by a system;
calculating a weight for a control values to be input to the system according to a comparison between the actual result and a specific range;
calculating the control value based on the actual result and the weight; and
controlling the target by inputting the calculated control value to the system.

18. The control method according to claim 17, wherein
a process of the calculating the weight includes calculating the weight corresponding to the actual result which is acquired this time based on a relationship between a past actual result and a past weight corresponding to the past actual result.

19. The control method according to claim 18, wherein
the relationship is a relationship in which the actual result at a predetermined time and a weight which is calculated based on the actual result for a predetermined period from the predetermined time are associated with each other.
